(19) United States
(12) Patent Application Publication
(10) Pub. No.: US 2023/0293260 A1
(43) Pub. Date: Sep. 21, 2023

(54) CONTAINMENT DEVICE

(71) Applicant: STELLENBOSCH UNIVERSITY, Western Cape Province (ZA)

(72) Inventor: Edward Joseph FUZY, Cape Province (ZA)

(21) Appl. No.: 18/008,121

(22) PCT Filed: Jun. 1, 2021
(86) PCT No.: PCT/IB2021/054784

§ 371 Date: Dec. 2, 2022

(30) Foreign Application Priority Data
Jun. 4, 2020 (GB) 2008435.6

(51) Int. Cl. A61B 90/40; A61G 10/00

(52) U.S. Cl. CPC A61B 90/40; A61G 10/005; A61B 2090/401

(57) ABSTRACT

A containment device includes a substantially transparent structure having a top, an open bottom with bottom edges aligned in a plane to form a planar base, and a plurality of sidewalls extending from the bottom to the top that define an interior for a patient’s head. A first sidewall has a pair of access openings for an operator’s hands, and an open end opposite the first sidewall is sized to accommodate at least a portion of the patient’s upper body when in use. The top includes a fist transparent panel extending from the first sidewall towards the open end at an upward incline relative to the plane (AA) of the base, the first transparent panel providing the operator with an undistorted and unobstructed view of the patient’s head.

### CROSS-REFERENCE TO RELATED APPLICATIONS

This application claims priority from United Kingdom patent application number 2008435.6 filed on 4 Jun. 2020, which is incorporated by reference herein.

### FIELD OF THE INVENTION

This invention relates to a containment device. More particularly, but not exclusively, this invention relates to a containment device for inhibiting the spread of disease.

### BACKGROUND TO THE INVENTION

Airborne diseases generally spread by aerosol transmission, contact transmission, or droplet transmission. Airborne diseases are caused by pathogens that are transmitted through the air. The relevant pathogens may be viruses, bacteria, or fungi, and they may be spread through breathing, talking, coughing, sneezing, raising of dust, spraying of liquids, or any activity which generates aerosolized particles. These particles are very small, generally smaller than 10 µm or smaller than 5 µm in size. These small particles may be referred to as an aerosol component, and they are so small that they become dispersed in the air. They are generally not gravity dependent and may linger in the air or accumulate in plumes around an infected individual. Masks may not be sufficiently effective at preventing the spread of diseases caused by these types of pathogens.

Airborne disease transmission is distinct from transmission by respiratory droplets. Respiratory droplets can vary in size, but they are usually large enough (greater than 5 µm, or greater than 10 µm) so that they fall to the ground rapidly after being produced. However, medical professionals and staff are at risk of being exposed to airborne diseases as well as respiratory droplets, especially when high risk airway manoeuvres such as intubations need to be performed.

At present, Personal Protective Equipment (PPE) such as facemasks, goggles and gloves are used to inhibit transmission. The extent of PPE used during medical procedures can vary, depending on the type of disease being treated, as well as on the preference of the medical professional. However, with the recent viral pathogen spread of the severe acute respiratory syndrome coronavirus 2 (SARS-CoV-2) commonly referred to as the Coronavirus or COVID-19, resources in PPE have become exceedingly limited. This is exacerbated by the fact that in many developing countries, the availability of PPE is, in any event, limited. In the era of COVI0-19, PPE and its role in the protection of clinicians has come to the forefront as an important medical technology.

In severe cases, COVID-19 patients or patients with respiratory failures may require high-risk airway manoeuvres such as intubation by laryngoscopy or even a tracheostomy, to provide supplemental oxygen to assist respiration. Intubation by laryngoscopy is usually done directly, with the medical professional physically observing the insertion of a laryngoscope into the patient’s mouth, and a ventilation tube that is inserted through the patient’s glottis into the trachea. Indirect laryngoscopy can be performed when a video-laryngoscope is available, and the medical professional then views the insertion by way of a video feed. Video-laryngoscopes are often not available, or in limited supply, so direct laryngoscopy is still used in most cases. If a tracheostomy is required, an emergency cut is created in the patient’s neck in order to place a ventilation tube directly into the patient’s windpipe or trachea.

Regardless of the type of high-risk airway manoeuvre performed, close proximity to the patient is still required which carries an inherent risk of infection to the medical professional or assistant when the patient has a respiratory disease such as COVID-19, influenza, tuberculosis, measles, chickenpox, etc. These high-risk airway manoeuvres are often difficult and need to be performed quickly and under stressful circumstances because the patient’s life may be at stake. With highly contagious patients it is common practice for these manoeuvres to be performed by a single medical professional, to limit infection risks. This can be both ineffective and dangerous since the help of an assistant may be required in many cases, and anyone in close proximity to the patient may be at risk of infection.

Available patient containment devices do not, in the applicant’s view, provide medical professionals with a clear, un-obstructed, undistorted, and close-up view of a patient, as well as permit ease of access to enable high-risk airway manoeuvres to be reliably performed.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

In accordance with the invention there is provided a containment device comprising:
a substantially transparent structure having a top, an open bottom and a plurality of sidewalls extending from the top to the bottom to define an interior for receiving a patient’s head, the open bottom having bottom edges aligned in a plane to form a planar base;
wherein a first sidewall has a pair of access openings for an operator’s hands, and the structure has an open end opposite the first sidewall, the open end sized to accommodate at least a portion of the patient’s upper body when in use;
characterized in that the top includes a first transparent panel extending from the first sidewall towards the open end at an upward incline relative to a plane of the base, the first transparent panel providing the operator with an undistorted and unobstructed view of the patient’s head.

The first transparent panel may be inclined at an angle of between 10 and 60 degrees, preferably between 20 and 50 degrees and most preferably between 25 and 45 degrees, relative to the plane of the base.

The top may include a second transparent panel extending from first transparent panel towards the open end at a downward incline relative to the plane of the base, the zone where the first and second transparent panels meet being at a maximum height of the structure.

A flexible barrier or curtain may be removably attachable to the open end of the structure to provide an at least partial seal between the patient’s upper body and the open end of the structure.

Access openings may be provided in at least two of the sidewalls. Each access opening may include an attachment rim thereat for fastening a medical glove at the access opening to extend therethrough. The attachment rims may include a recessed peripheral surface for removably receiving a proximal end of the medical glove to fasten it at the access opening. The access opening may include a fastening ring which magnetically attaches to the access opening to hold the glove in place, and a cover which magnetically attaches to the access opening in the event that the opening is to be blocked.

In some embodiments, the containment device is provided with a base tray which attaches to the base. The base tray may have a depth to accommodate a mattress, such as a theatre table mattress, and may have a bottom plate shaped to extend beneath the mattress. The bottom plate may have a U-shape such that it extends only part-way beneath the mattress so as not to interfere with an attachment of the mattress to a support surface thereof, such as hook and loop fasteners that may be present.

The base may include a flange in the plane of the base, and the base tray may have a cooperating upper flange. The flanges of the base and base tray may magnetically attach together.

One or more of the sidewalls may include an access doorway that is open to the bottom. The access doorway may be covered by an access flap and may be sized to allow equipment such as a ventilator tube to pass therethrough with the access flap opening to uncover access doorway the as the containment device is placed over the head of a patient. This has the advantage that ventilator tubing does not need to be disconnected when placing the containment device over the patient’s head.

The containment device may include a suction port extending from the structure for operatively extracting air from the interior of the structure. The suction port may be provided on the top of the structure, and an air filter may be provided adjacent the suction port.

The flexible barrier may, in some embodiments, include one or more magnets or magnetic material near its periphery, for removably attaching the flexible barrier to the open end of the structure. The open end may be provided with a cooperating magnet or magnetic material.

Embodiments of the invention will now be described, by way of example only, with reference to the accompanying drawings.

### BRIEF DESCRIPTION OF THE DRAWINGS

In the drawings:

FIG. 1 is a three-dimensional view of a first example embodiment of a containment device;

FIG. 2 shows the device of FIG. 1 from a different angle;

FIG. 3 is a side elevation of the device of FIG. 1;

FIG. 4 is a three-dimensional view showing the device of FIG. 1 together with a base tray;

FIG. 5 shows the containment device with medical gloves and a removable barrier or curtain attached;

FIG. 6 shows the containment device in use with a patient being attended to by an operator and an assistant;

FIG. 7A shows a detail view of an access doorway in a first sidewall of the containment device;

FIG. 7B is similar to FIG. 7A and shows the access doorway open;

FIG. 8 shows access openings of the containment device in more detail;

FIG. 9A shows a fastening ring being attached to an attachment ring of the access opening;

FIG. 9B is similar to FIG. 9A and shows the fastening ring attached;

FIG. 10 is a three-dimensional view of a second example embodiment of a containment device;

FIG. 11 shows the device of FIG. 10 from a different angle;

FIG. 12 is a three-dimensional view of a third example embodiment of a containment device;

FIG. 13 shows the containment device of FIG. 12 in use;

FIG. 14 is a three-dimensional view of a fourth example embodiment of a containment device; and

FIG. 15 shows the device of FIG. 14 from a different angle.

### DETAILED DESCRIPTION WITH REFERENCE TO THE DRAWINGS

There is provided a cover, chamber, or containment device that may be used during medical procedures. The containment device may also be referred to as a dangerous airway containment chamber or an intubation chamber. A plurality of glove ports or access openings are provided for a clinician to perform tasks inside the cover or device. Rims, attachment rims or sleeves may be provided to hold gloves such as medical gloves that extend into an inside region of the device. The containment device or cover may act as a seal and it may at least partially isolate or contain air or fluid inside the device while an airway manipulation or other medical procedure is performed inside the device. Air may be drawn out of the device through a filter and a partial vacuum or negative pressure may be created inside the device. This may inhibit any dangerous, contagious or harmful particles, pathogens, nanoparticles or the like from escaping from within the device. A pliable member or barrier may be provided and is releasably attachable to an open side of the device. The device may be shaped such that an operator has an undistorted and unobstructed view of the patient. A top transparent panel may be provided forming the primary viewing panel, and such panel may be provided at an angle relative to horizontal, such as an upwardly sloping angle.

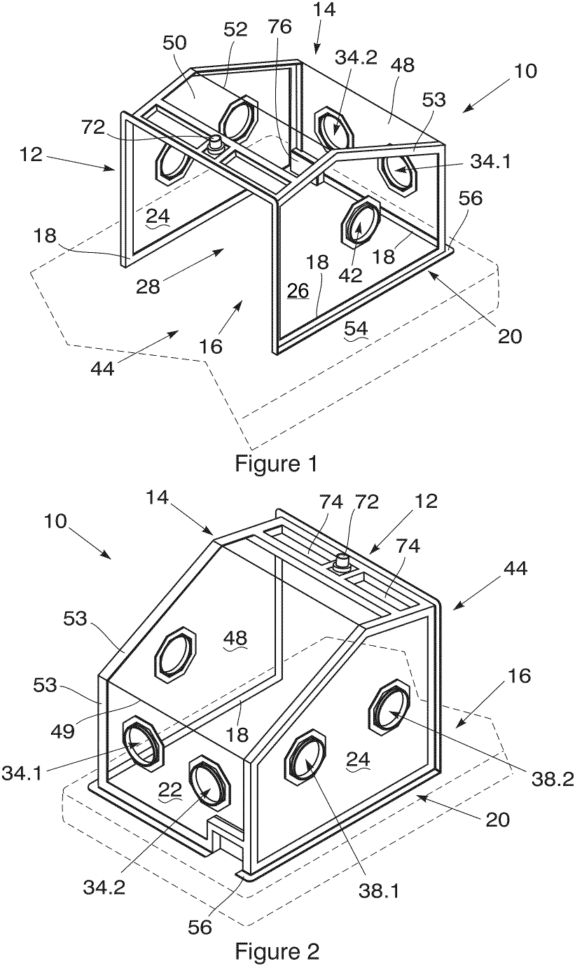

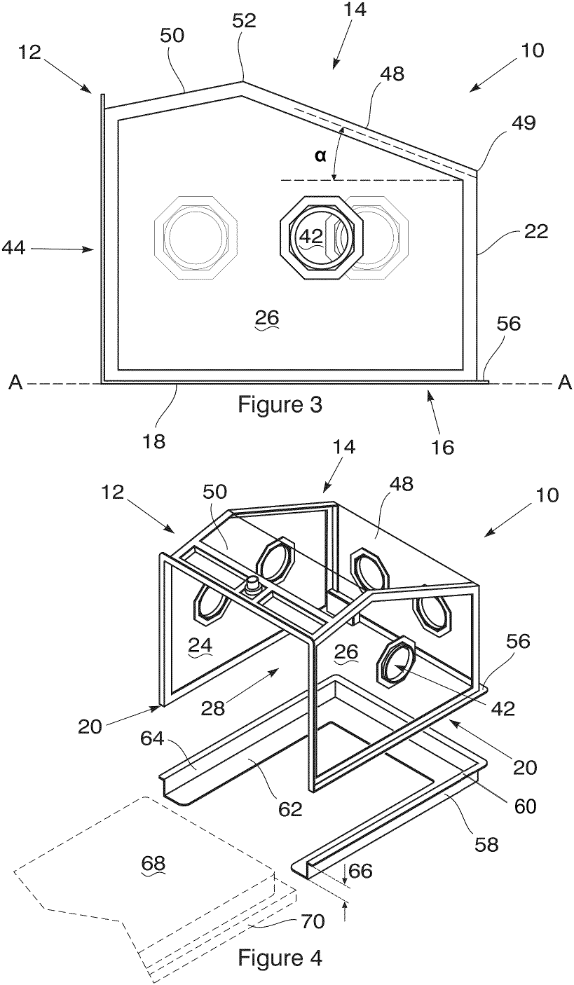

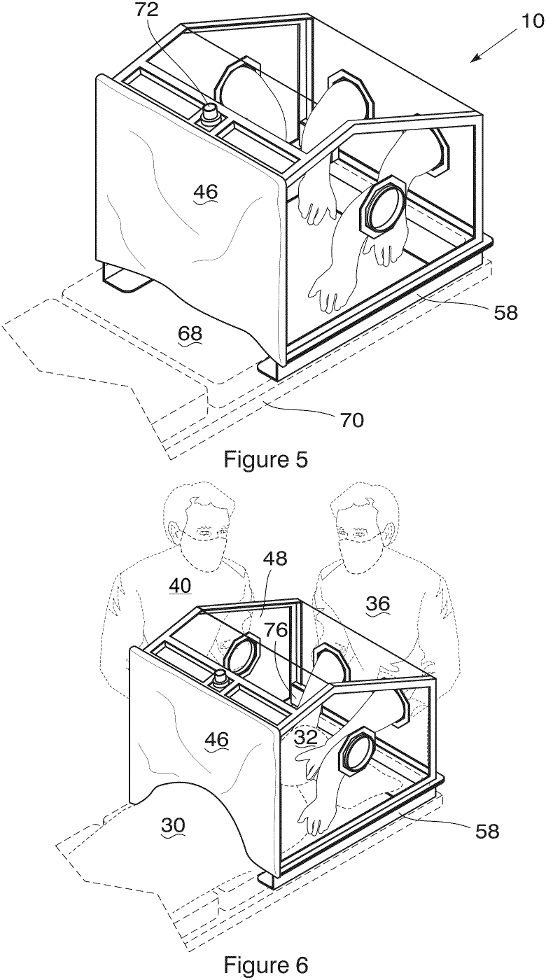

FIGS. 1 to 6 show an example embodiment of a containment device (10) according to the present disclosure. The containment device (10) may include a substantially transparent structure (12) having a top (14), an open bottom (16) and a plurality of sidewalls (22, 24, 26) that extend from the top (14) to the bottom (16). The bottom (16) may include bottom edges (18) that are aligned in a plane (A-A, shown in FIG. 3), to form a planar base (20). The sidewalls (22, 24, 26) and top (14) define an interior (28). As shown in FIG. 6, the interior (28) may receive a part of a patient (30), such as the patient’s head (32).

In the illustrated embodiment the sidewalls include a first sidewall (22), which may also be termed a cranial sidewall as it is on a cranial side of the patient (30) when in use, a right sidewall (24) (adjacent the patient’s right shoulder) and a left sidewall (26) (adjacent the patient’s left shoulder). The right and left sidewalls (24, 26) may be disposed at generally right angles to the first sidewall (22), and all three sidewalls may be generally vertical relative to the plane (A-A) of the base (20). The first sidewall (24) may have a pair of access openings (34.1, 34.2) for the arms and hands of an operator (36) to extend through. The operator (36) may be a clinician who is required to perform an airway operation on the patient (30), such as an intubation. The other sidewalls (24, 26) may also optionally have access openings therein. For example, the right sidewall (24) may have a pair of access openings (38.1, 38.2) through which the arms and hands of an assistant (40) may extend as shown in FIG. 6. The left sidewall (26) is shown, in this embodiment, with a single access opening (42) which may optionally be used by a second assistant (not shown).

The structure (12) includes an open end (44) that is opposite the first sidewall (22). The open end is sized to accommodate a portion of an upper body of the patient (30) and may also be referred to as a caudal end, and it may face the patient’s feet during use. A barrier (46) or curtain is provided at the open end (44) to provide an at least partial seal between the patient (30) and the open end (44) of the structure. The barrier (46) may be flexible and may be removable from the device (10). The barrier (46) may be a flexible membrane such as a non-porous drape or a surgical drape or torso drape, a curtain, or a flexible membrane made from rubber, latex, nylon, synthetic material, plastics, polymers, fabric, cotton, woven materials, nonwoven materials or nonwoven fabric, or combinations of these materials, or any flexible material able to conform to the patient’s body. The flexible barrier (46) may be made from a flexible air tight material. The flexible barrier may be made from a material such as a fine mesh of synthetic polymer fibres or nonwoven polypropylene fabric. The flexible barrier may also be partially air tight, and it may be made to comply with an air filtration standard, for example the N95 standard requiring it to filter at least 95% of airborne particles. Other filtration standards may also be used as required.

The top (14) includes a first transparent panel (48) which forms a primary viewing window for the operator (36) to see the patient’s head (32). The first transparent panel (48) extends from a top end (49) of the first sidewall (22) towards the open end (44) at an upward incline relative to the plane of the base (A-A) and therefore relative to horizontal when the base is placed on a horizontal surface such as a mattress or theatre table. The angle (α) of incline is shown in the side view of FIG. 3.

The applicant has found that providing the first transparent panel (48) at an upward incline has several advantages. The first transparent panel (48) can be made large and flat so as to extend across the majority of the top (14). Visual distortion from the viewpoint of the operator (36) that occurs where the first transparent panel (48) meets other components (such as the top end (49) of the first sidewall (22)) is therefore reduced. Distortion may also be reduced because the operator (36) looks through the first transparent panel (48) towards the patient’s head (32) at generally right angles through the panel. Reflection from overhead lights (not shown) may also be reduced with the upwardly inclining first transparent panel (48) when compared to horizontal transparent surfaces. The upwardly inclined first panel (48) allows the operator to get closer to the patient’s head on the cranial side of the head (32) while providing additional height towards the open end (44). The additional height facilitates ease of placing the device (10) over the patient (30) while potentially preventing the patient from becoming unnecessarily claustrophobic or otherwise distressed.

The angle of incline (α) of the first transparent panel (48) may vary depending on the overall height of the device (10), the height of the bed or other surface on which the patient (30) is expected to lie, and the expected position of the operator (36) relative to the patient (30). Generally the incline angle (α) will be between 10 and 60 degrees relative to the plane of the base (20). In some embodiments, the incline angle (α) may be between 20 and 50 degrees, and may, for example, be between 25 and 45 degrees.

The top (14) may include a second panel (50), which may optionally also be transparent, that extends from the first transparent panel (48) towards the open end (44) at a downward incline relative to the plane (A-A) of the base (20). The zone (52) or line where the first and second panels (48, 50) meet may be at a maximum overall height of the structure (12), as best shown in FIG. 3.

The structure (12) (including the sidewalls and top) of the containment device (10) may be made from a rigid material which may be substantially transparent, or at least translucent. The rigid material may be any one of glass, plastic or polymeric material such as Perspex™, acrylic resins, combinations of these materials, or any other rigid, substantially transparent and airtight material. In other embodiments, the transparent material may be flexible and held in place by a rigid frame.

Bioprocessing Equipment (BPE) cladding may be used. The structure (12) may include a plurality of seams which may be joined by plastics welding, adhesives or the like. The seams or other parts of the structure may be provided with reinforcing members (53) which may provide structural stability. The substantial transparency of the structure, including its sidewalls, top and panels may enable the operator or assistant to have a field of view into the interior of the device. This may be advantageous in case of difficulties often encountered in the intubation of a patient, and it may enable a clear view of the patient’s head. It may also inhibit any area of the patient’s head from being obstructed from direct vision during the procedure. The reinforcing members (53) may collectively form a frame or cage to which the structure may be fastened, or the frame or reinforcing members may form part of the structure. The reinforcing members (53) may be made from any rigid material, such as metals, aluminium, steel, stainless steel, extrusion moulded plastics, other plastics, polymers, composites, wood, or any other rigid material or combinations of materials. The reinforcing members may form a reinforced cage or frame which may be cladded by the sidewalls and by one or more top panels or roof panels. In other words, the structure (12) may include the frame or cage, or the structure (12) may be fastened or attached to the frame or cage.

The device (10) can be placed directly onto a surface such as a bed (54) on which the patient (30) may be lying, as illustrated in FIGS. 1 and 2, such as a hospital bed. In the illustrated embodiment, the base (20) has a flange (56) extending in the plane (A-A) of the base (20). The flange (56) may form part of the base (20) and provide a flat, narrow surface for the device (10) to rest directly on the surface such as the bed (54) shown in FIGS. 1 and 2. Medical mattresses are particularly soft, and some of these mattresses are sophisticated devices that can inflate and deflate in a controlled way, for example to inhibit pressure points in patients that are unable to roll around. The flange of the base may therefore be wide enough (for example about 70 mm wide) to inhibit damage to the mattress, and so as to distribute force more evenly along the surface of the mattress, while also providing stability to the structure of the containment device. The flange (56) may also facilitate a seal between the device (10) and the bed (54) or other surface on which the device is placed in use.

The device (10) may also be provided with a base tray (58) as illustrated in FIGS. 4 to 6. The base tray (58) may attach to the base (20) in a releasable manner. The base tray (58) may have an upper flange (60) which cooperates with the flange (56) of the base (20) to locate the flanges together. Magnets may be provided on either or both of the base tray flange (60) or the base flange (56) for this purpose. The base tray (58) may have a U-shaped bottom plate (62), and sidewalls (64) extending from the bottom plate (62) to the tray flange (60) and defining a depth (66) of the base tray (58). The depth (66) of the base tray (58) may be selected to accommodate a mattress (68), such as a mattress of a theatre table (70), with the bottom plate (62) extending only part-way beneath the mattress (68). Having the bottom plate (62) extend only part-way beneath the mattress (68) may be important so as not to interfere with an attachment of the mattress (68) to the theatre table (70), such as via hook and loop fasteners (not shown) that may be present.

By using the base tray (58) in conjunction with relatively narrow beds such as theatre tables (70), the device (10) is held securely in place with the height of the base (20) at about the same level as when the device is simply placed on top of relatively wider beds (54) such as hospital beds. The base tray (58) therefore allows the same structure (12) to be used in a versatile manner in different settings. The base tray may be made of varying dimensions that can adapt the containment device to fit emergency room stretchers, or ICU pressure mattresses, or the base plate and containment device may be placed directly onto theatre tables. The base tray may be relatively inexpensive to manufacture. The base plate may provide the advantage that it may limit the need to vary the size of the structure of the containment device for varying applications. In other words, the size of the containment device or the size of the open end of the structure may be standardised, and various sizes of base trays may be made depending on the particular application.

The containment device (10) preferably includes a suction port (72) by which the air can be operatively extracted from the interior (28) of the structure (12). The suction port (72) is illustrated in the top (14) in this embodiment and has an air filter (74) provided around the suction port (72). As best shown in FIG. 2, the air filter (74) in this illustration is a membrane placed on an underside of a shallow chamber formed in the top (14) and which is connected to the suction port (72). The suction port (72) could, however, be provided in any other part of the structure (12) including in any sidewall, and one or more filters may be provided at the suction port (72) or downstream of it. A suction tube (not shown) is attached to the suction port and then to a vacuum pump for extracting air through the suction tube, thereby generating a negative pressure in the interior. Downstream of the pump, the air could be filtered again, or it could be vented to the outside or dealt with in any appropriate manner as is known. The filter or filters may be operable to filter out droplets, aerosolized pathogens or any other dispersed particles from extracted air and may include a high-efficiency particulate absorbing (HEPA) filter.

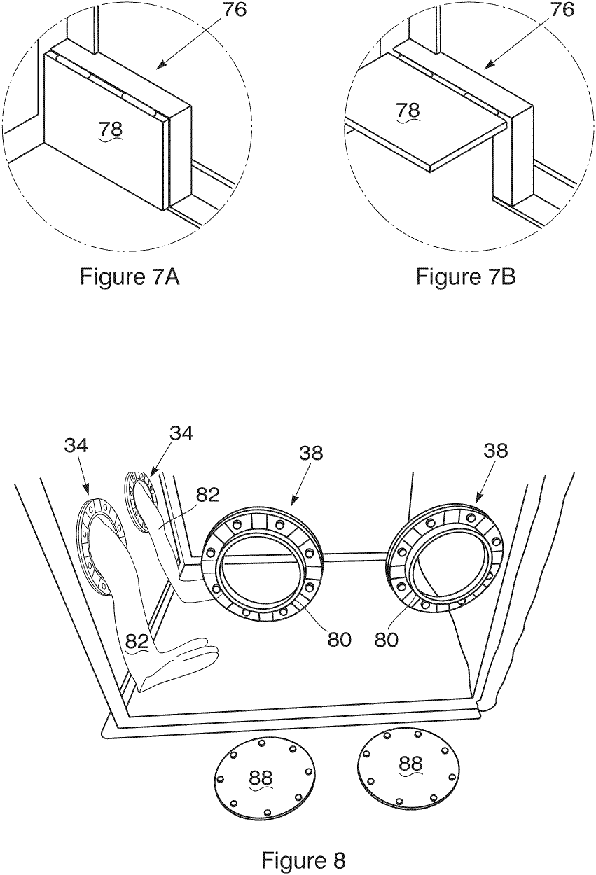

One or more of the sidewalls may have an access doorway (76) that is open to the bottom (16). In this embodiment the access doorway (76) is in the first sidewall (22) that is closest to the operator (36) when in use. The access doorway (76) is shown in more detail in FIGS. 7A and 7B. The access doorway (76) may be covered by an access flap (78) which optionally attaches to the access doorway (76) releasably such as through the use of magnets. The access doorway (76) is sized to allow equipment such as a ventilator tube (not shown) to pass therethrough with the flap opening to uncover the access doorway as the containment device (10) is placed over the head (32) of a patient. This has the advantage that the equipment such as the ventilator tubing does not need to be disconnected and fed through a separate access port of the device (10); it is simply received in the doorway when the device is placed over the patient (30). Other arrangements of access flaps, doorways, or curtains that achieve that same objective and have a slot, doorway or passage open to the bottom may also be provided.

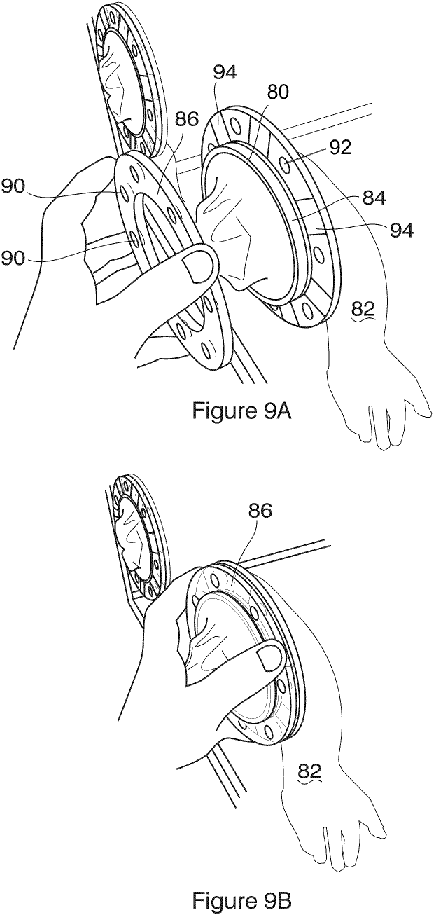

FIGS. 8, 9A and 9B show the access openings (34, 38) in more detail. Each access opening (34, 38) may include an attachment rim (80) thereat for fastening a medical glove (82) at the access opening to extend therethrough. The attachment rim (80) may include a recessed peripheral surface (84) for removably receiving a proximal end of the medical glove to fasten it at the access opening. A fastening ring (86) may be provided which attaches to the access opening to hold the glove (82) in place. The fastening ring (86) may have radially dispersed magnets (90) (such as neodymium magnets), magnetic material or other fasteners. In this embodiment, the magnets (90) cooperate with similar magnets (92) on the attachment rim (80). In this illustration, the attachment rim (80) includes an inner ring (94) fastened to an inner surface of the relevant sidewall. One or more covers (88) in the form of a lid or disc with cooperating magnets or fasteners may also be provided, that may attach to the access opening to close or block it when it is not being used.

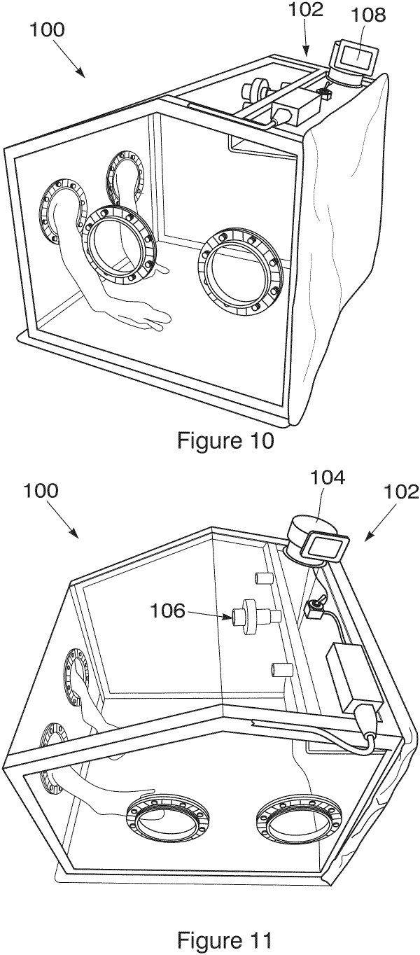

FIGS. 10 and 11 show a further embodiment of a containment device (100). This device (100) is similar to the device (10) of FIGS. 1 to 9 but includes a different arrangement which includes a contained extraction module (102) to control air exchange and negative pressure inside an interior of the device (100). The contained extraction module (102) may be referred to as a “dirty chamber”. A vacuum pump (104) may be provided to extract air from the interior of the device through one or more filters (106) and into the contained extraction module. A further filter and/or a unidirectional valve may be provided at or near the outlet (108), for example to inhibit any “dirty” or contaminated air, dispersed particles or pathogens from escaping the contained extraction module.

It will be appreciated that the contained extraction module may be attached to the top of the structure of the containment device, or a separate extraction module may be provided which may include an enclosed or self-contained chamber or container connectable to a conduit or suction tube and/or to the suction port. The separate container of the contained extraction module may be provided external to the interior of the containment device. It may for example be possible that the suction port does not need to extend through the structure. For example, a conduit may be provided with an open end or suction port provided somewhere in the interior of the structure, and the conduit may then pass through the open end of the structure (adjacent to the flexible barrier, or even through the flexible barrier) towards the exterior of the structure. In such an embodiment, the suction port may extract air from the interior of the structure (optionally through a filter) and into the separate contained extraction module which may be attached to the conduit somewhere outside the containment device.

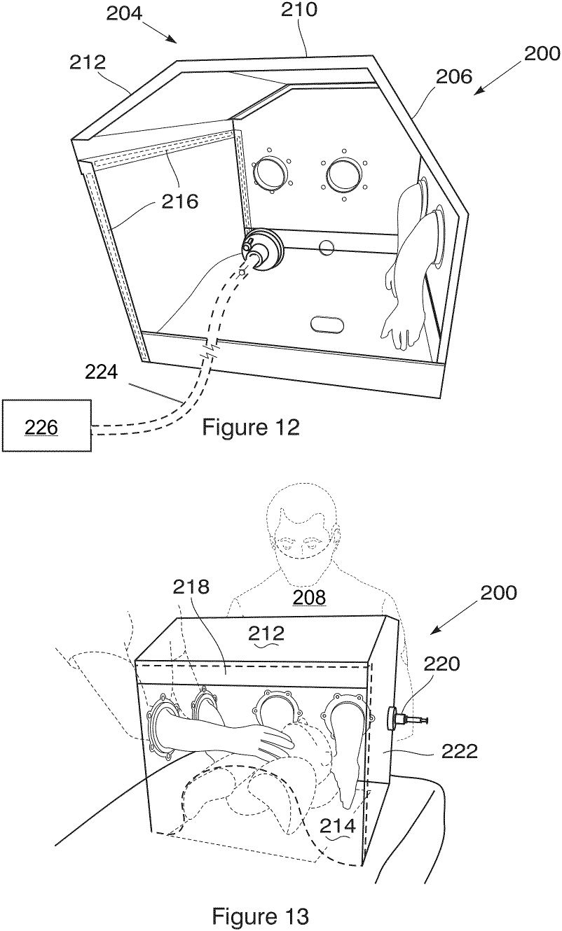

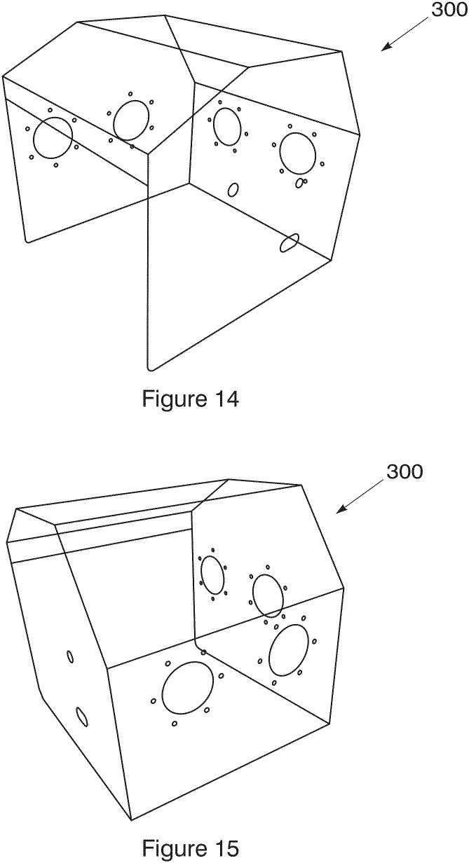

FIGS. 12 and 13 show a further embodiment of a containment device (200). This device (200) has a top (204) that is formed of three panels: a first transparent panel (206) that forms the main viewing panel for the operator (208), a second transparent panel (210) and a third transparent panel (212). This device (200) also includes a barrier (214) that attaches to the device by means of magnetic strips (216) provided at the open end, including along a skirt (218) at a top of the open end. A suction port (220) is provided in a left sidewall (222) of the device (200), rather than on the top (204). An associated suction tube (224) and extraction module (226) or vacuum pump are also shown.

FIGS. 14 and 15 show a yet further embodiment of a containment device (300), that is similar to the device (200) of FIGS. 12 and 13 but is made of transparent sheets that have been glued, ultrasonically welded or otherwise connected together along their edges without using reinforcing members.

The containment device may be portable so that it may operatively be positioned over the patient’s head with the patient in a supine position, the patient’s upper body extending through the open end of the structure.

Some or all of the medical gloves and attachment rims may be pre-installed before the operative positioning so as to facilitate quick positioning and use of the containment device. This may be advantageous when the operator or assistant needs to act quickly, for example in an emergency scenario, when a patient with a contagious disease requires treatment such as intubation by laryngoscopy. The containment device may be particularly useful when the patient has a contagious respiratory disease or airborne disease. Preferably, the containment device may be sterilised before, and after each use. Prior to use, each of the attachment rims may have a medical glove fastened thereto, so that the containment device may be used quickly after it is positioned over the patient’s head. The medical gloves may be single-use medical gloves that may be disposed of and replaced after each use of the containment device. The attachment rims may also be permanently fastened to the structure of the containment device, or may be removably fastened thereto, and the medical gloves may be pulled out and removed from the attachment rims once the operator and assistant have performed the medical procedure inside the device. The medical gloves may be discarded after use, and replaced with new ones before a subsequent use of the containment device. Preferably, the gloves may be left attached or fitted to the attachment rims in the access openings for a selected time period, while the vacuum pump continues to extract air and any dispersed pathogens from the interior. A time period of a few minutes may be sufficient, however, it may be required for the suction device or vacuum pump to continue extraction for 5 minutes or more, 10 minutes or more, or 15 minutes or more, depending on the type of disease. The chamber itself may be sterilized prior to each use.

The attachment rims may facilitate safe removal of the medical gloves from the interior of the containment device. The proximal end of the glove may act as an O-ring, seal or gasket, and any pathogens or viral particles that may be on an outer surface of the glove inside the interior may be kept or contained in the medical glove when it is removed. The medical gloves may be removed from the access openings by pulling them out through the access openings, and inverting the gloves in the process, to contain any pathogens or the like inside the glove. This may prevent any droplets or particles from splashing or being flung into the air, thereby protecting any users of the containment device. The medical gloves may be relatively inexpensive and easy to replace gloves, or “off the shelf” gloves that may be readily available. This may provide advantage in that readily available gloves may be retro-fitted to the attachment rims while providing a sealed connection. The substantially air tight seal between the medical gloves and the attachment rims may provide protection against pathogens or dispersed particles inside the interior of the structure of the containment device.

In use, the operator or assistant may position the device over the patient’s upper body and then attach the flexible barrier to the open end of the structure. The flexible barrier may be weighted, or it may have a substantial weight, so that it may conform to the shape of the patient’s body. Once the flexible barrier is attached, the containment device may be at least partially sealed, and the operator may use their hands inside the medical gloves as needed. The operator may stand at the cranial sidewall behind the top of the patient’s head, or at one of the other sidewalls. The containment device may protect the operator, assistant or any bystanders of any harmful pathogens or airborne particles that may be dispersed inside the interior. A vacuum pump may be provided as described above and it may be switched on to extract air and any dispersed particles from inside the interior, and a negative pressure may be created. Once intubation or other high-risk airway manoeuvre (or any other medical procedure on the upper body of the patient) is then performed, the patient may for example be ventilated, and the vacuum pump may continue to provide negative pressure inside the interior. It will further be appreciated that even though an at least partial seal is created between the flexible membrane and the patient’s body, the negative pressure inside the interior may urge any air, pathogens or dispersed particles to migrate towards the suction port(s) or outlet(s) to be extracted or filtered out safely.

The present disclosure may provide the advantage that it may provide a clear and direct line of sight to the operator during a dangerous airway manoeuvre or other medical procedure. Performing direct laryngoscopy is often required when video-laryngoscopes are not available, and the operator (or assistant) may be required to stand very close to the patient’s head. The sloping first transparent panel of the top of the structure at the angle of incline enables the operator to have a direct line of sight into the patient’s mouth and throat, while allowing the operator to get closer to the patient’s head, and it may also prevent or inhibit light to be reflected into the operator’s or into the assistant’s eyes, as described herein. High-powered lights are often used in medical environments, which may inhibit an operator’s vision if it should be reflected into his or her eyes. The present disclosure may facilitate light to be deflected or dispersed away from the eyes of the operator or assistant. The present disclosure may also inhibit the “burn” (rate of usage) of personal protective equipment (PPE), because if the containment device is used, less PPE (e.g. facemasks, visors, facial splash covers, goggles etc.) may be required. The containment device of the present disclosure may be cleaned after each use and reused. The medical gloves may be replaced, and the device and components may be sterilized or cleaned before reuse. Alternatively, the entire device may be discarded after each use. Any filter(s) may also be removed and replaced after each use.

A further advantage may be provided by having two access openings for the assistant. This may be particularly advantageous during dangerous medical procedures such as a tracheostomy. Two surgeons or medical professionals may work on the patient during such an operation, while the interior of the containment device may still be kept under negative pressure. The size and position of the access openings or access ports may be about 110 mm in diameter (or about 100 mm) to allow ergonomic placement of the operator or assistant’s arms and hands. The attachment rims may also be made deep enough to facilitate the operator or assistant to rest their upper arms thereon during use. The access openings may be spaced wide enough from one another, to facilitate the medical procedure to be performed in as naturally as possible position. The access openings may be spaced to enable range of movement of the operator’s or assistant’s hands during the medical procedure.

The present disclosure may provide protection against both coarse and fine respiratory droplets. Even airborne pathogens, virulent matter or aerosolized particles that are “floating” or dispersed in the air may be contained and safely extracted by the containment device described. The present disclosure may provide protection during a “peri-intubation” period, for example during airway manipulation. The present disclosure may to contain, control, or inhibit the dispersion of infective fluids or particles, and air may be extracted safely to mitigate infective potential or the spread of disease. The containment device of the present disclosure may be portable and easy to position quickly over a patient’s head, for example during an emergency. Intubation of patients generally need to be performed very quickly, typically in minutes or even less. The containment device may facilitate this to be done as quickly and as safely as possible. It may also alleviate or prevent the need for a dedicated quarantine room. The vacuum pump may in some instances continue to operate, providing a portable sealed containment device. The containment device may be sealed within selected tolerances. The vacuum pump may also be portable and connected to the containment device.

The containment device may have a generally cuboid shape. However, many other shapes are possible, such as curved shapes, domed shapes, angled shapes, rectilinear shapes, polyhedral shapes etc. The various edges of the device may also be curved or angled. The transparency of the sidewalls, the top panels, the lip or caudal panel, and the top of the device may facilitate a clear view of the interior. It will further be appreciated that even though the present disclosure may provide advantages for patients with severe acute respiratory syndrome coronavirus 2 (SARS-CoV-2), commonly referred to as COVID-19, patients with other types of respiratory disease or any other contagious disease may be treated with the aid of the containment device disclosed. The present disclosure may enable the operator and the assistant to use their hands in the interior at the same time, which may be advantageous in many types of medical procedures.

The foregoing description has been presented for the purpose of illustration; it is not intended to be exhaustive or to limit the invention to the precise forms disclosed. Persons skilled in the relevant art can appreciate that many modifications and variations are possible in light of the above disclosure. The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention, which is set forth in the following claims.

Finally, throughout the specification and accompanying claims, unless the context requires otherwise, the word ‘comprise’ or variations such as ‘comprises’ or ‘comprising’ will be understood to imply the inclusion of a stated integer or group of integers but not the exclusion of any other integer or group of integers.

What is claimed is:

1. A containment device comprising:
a substantially transparent structure having a top, an open bottom and a plurality of sidewalls extending from the top to the bottom to define an interior for receiving a patient’s head, the open bottom having bottom edges aligned in a plane to form a planar base,
wherein a first sidewall has a pair of access openings for an operator’s hands, and the structure has an open end opposite the first sidewall, the open end sized to accommodate at least a portion of the patient’s upper body when in use, and
wherein the top includes a first transparent panel extending from the first sidewall towards the open end at an upward incline relative to the plane of the base, the first transparent panel providing the operator with an undistorted and unobstructed view of the patient’s head.

2. The containment device of claim 1, wherein the first transparent panel is inclined at an angle of between 10 and 60 degrees relative to the plane of the base.

3. The containment device of claim 1, wherein the top includes a second transparent panel extending from the first transparent panel towards the open end at a downward incline relative to the plane of the base, and a zone where the first and second transparent panels meet being at a maximum height of the structure.

4. The containment device of claim 1, further comprising, a flexible barrier removably attachable to the open end of the structure to provide an at least partial seal between the patient’s upper body and the open end of the structure.

5. The containment device of claim 1, wherein access openings are provided in at least two of the sidewalls.

6. The containment device of claim 1, wherein each access opening includes an attachment rim thereat for fastening a medical glove at the access opening to extend therethrough.

7. The containment device of claim 6, wherein each attachment rim includes a recessed peripheral surface for removably receiving a proximal end of the medical glove to fasten it at the access opening.

8. The containment device of claim 6 wherein each access opening includes a fastening ring magnetically attached to the access opening to hold the glove in place, and a cover magnetically attached to the access opening when the opening is blocked.

9. The containment device of claim 1, further comprising, a base tray attached to the base, the base tray having a depth to accommodate a mattress and having a bottom plate shaped to extend beneath the mattress.

10. The containment device of claim 9, wherein the bottom plate of the base tray has a U-shape such that it extends only part-way beneath the mattress so as not to interfere with an attachment of the mattress to a support surface thereof.

11. The containment device of claim 9, wherein the base includes a flange in the plane of the base, the base tray having an upper flange, and the flanges of the base and base tray magnetically attached together.

12. The containment device of claim 1, further comprising, an access doorway covered by an access flap in one of the sidewalls adjacent the open bottom, the access doorway sized to allow a tubing to pass therethrough with the flap opening to uncover the access doorway as the containment device is placed over the head of the patient without disconnecting the tubing when placing the containment device over the patient’s head.

13. The containment device of claim 1, further comprising, a suction port extending from the structure for operatively extracting air from the interior of the structure.

14. The containment device of claim 13, wherein the suction port is provided on the top of the structure and an air filter is provided adjacent the suction port.

15. The containment device of claim 4, wherein the flexible barrier includes a magnet or magnetic material near its periphery, for removably attaching it to the open end of the structure, the open end including a cooperating magnet or magnetic material.

16. The containment device of claim 1, wherein the first transparent panel is inclined at an angle between 20 and 50 degrees relative to the plane of the base.

17. The containment device of claim 1, wherein the first transparent panel is inclined at an angle between 25 and 45 degrees relative to the plane of the base.

18. The containment device of claim 7, wherein each access opening includes a fastening ring magnetically attached to the access opening to hold the glove in place, and a cover magnetically attached to the access opening when the opening is blocked.

19. The containment device of claim 10, wherein the base includes a flange in the plane of the base, the base tray having an upper flange, and the flanges of the base and base tray magnetically attached together.
